(19) United States
(12) Patent Application Publication
(10) Pub. No.: US 2020/0061859 A1
(43) Pub. Date: Feb. 27, 2020

(54) Razor

(71) Applicant: JonMichael Harmon, Santa Cruz, CA (US)

(72) Inventor: JonMichael Harmon, Santa Cruz, CA (US)

(21) Appl. No.: 16/551,841

(22) Filed: Aug. 27, 2019

Related U.S. Application Data
(60) Provisional application No. 62/723,072, filed on Aug. 27, 2018.

(51) Int. Cl. B26B 21/40; B26B 21/56; A46B 9/04; A61M 35/00; A61B 17/34

(52) U.S. Cl. CPC B26B 21/4018; B26B 21/56; A46B 2200/1066; A61M 35/003; A61B 17/34; A46B 9/04

(57) ABSTRACT

This non-provisional utility patent application contains several innovations to a razor used for shaving the face or any part of the body, as well as other hygiene products.

1. To give a very smooth shave a razor can be made to with one or more guard/s between the razor blades. These guards will lighten the pressure of the blades to make the shave more comfortable.

2. To make it easier for hairs in a razor to wash out, the razor blades can rotate to parallel. This gives the blades more space between each other making it easier for water to wash the hairs away.

3. A razor with curved blades will make it easier to cut through hair.

4. A “v” shaped brush can be made to better clean the places between one's teeth.

5. A toothbrush's head can be shaped to fit around the tooth where bristles are placed at various angles to ergonomically surround the tooth. This allows someone to brush all sides of their teeth at once.

6. A toothbrush with a built-in place to hold floss, or to hold a brush that cleans in-between the cracks in the teeth.

7. Toothbrush with a swivel will help the toothbrush clean more accurately. Said swivel can be on the handle or between the head and handle.

8. Some people wear teeth straightening covers for many hours of the day. However, they could be multi-tasking while wearing these retainers. Teeth whitening ingredients could be put inside the retainer so while they wear it their teeth get whitened and straightened at the same time. Teeth whitening paste that is made with little or no fluoride so it may be applied to the inside of a retainer. A tooth whitening flat or shaped layer can be used on the inside of a retainer and even shaped to fit into the retainer by use of a groove snap or ergonomic fitting. A retainer can be specifically made to fit with said whitening layer.

9. A device can be made to pop pimples by the force of a circular part. The pimple can be inside of said circular part and when the tool is pressed the pimple will get popped.

10. Acne prevention cream with the active ingredient Chlorhexidine gluconate with foaming and/or exfoliating ingredients.

### DESCRIPTION OF ILLUSTRATION

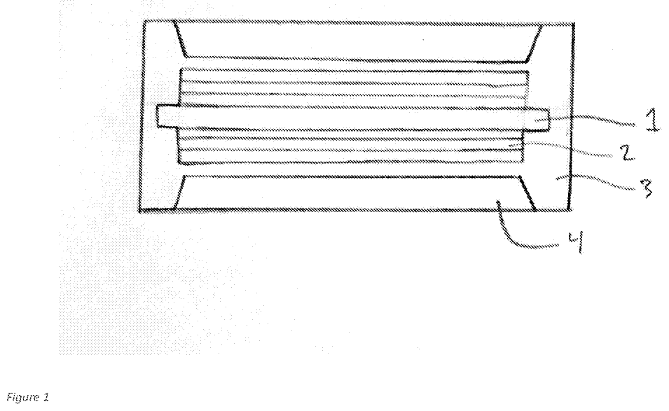

FIG. 1 shows a razor with a guard between the blades.

The guard between the razor blades, (1) has razor blades (2) on either side of it. The razor head (3) has outside guards (4) on two of its edges.

What is claimed is:

1) An apparatus comprising:
razor blades, a head where said razor blades are place; and a handle that is connected to said head.

2. As described in claim 1, a razor with one or more guards between the razor blades, where the razor blades face in the same general direction.

3. As described in claim 1, a razor where the razor blades can rotate.

4. As described in claim 1, said razor blades can be curved.

4) An apparatus comprising a teeth cleaning or whitening component.

5. As described in claim 4, a toothbrush where there is a protrusion of the head along the line of axis that runs parallel or close to parallel with the toothbrush handle. Or the bristles along said axis are longer and/or stiffer.

6. As described in claim 4, a toothbrush's head can be shaped to fit around the tooth where bristles are placed at various angles to ergonomically surround the tooth.

7. As described in claim 4, a toothbrush with a built-in place to hold floss, or to hold a brush that cleans in-between the cracks in the teeth.

8. As described in claim 4, a toothbrush where the head can be detached and replaced with another head that is a different design.

9. As described in claim 4, a toothbrush comprising a swivel between the head and handle.

10. As described in claim 4, a tooth whitening paste that is made with little or no fluoride so it may be applied to the inside of a retainer. A tooth whitening flat or shaped layer can be used on the inside of a retainer and even shaped to fit into the retainer by use of a groove snap or ergonomic fitting. A retainer can be specifically made to fit with said whitening layer.

11) An apparatus comprising a pimple reducing component.

12. As described in claim 11, a device that pops pimples using a circular or close to circular part.

13. As described in claim 11, an acne prevention cream with the active ingredient Chlorhexidine gluconate with foaming and/or exfoliating ingredients.
